(19) United States
(12) Patent Application Publication
(10) Pub. No.: US 2014/0288380 A1
(43) Pub. Date: Sep. 25, 2014

(54) IRIS RETRACTOR ASSEMBLIES

(71) Applicant: APX OPHTHALMOLOGY LTD., Haifa (IL)

(72) Inventors: Ehud Assia, Tel Aviv (IL); Nir Lilach, Kfar Yehoshua (IL); Eliahu Eliachar, Haifa (IL)

(73) Assignee: APX OPHTHALMOLOGY LTD.

(21) Appl. No.: 14/353,306

(22) PCT Filed: Oct. 24, 2012
(86) PCT No.: PCT/US2012/061513

§ 371 Date: Apr. 22, 2014

Related U.S. Application Data
(60) Provisional application No. 61/550,518, filed on Oct. 24, 2011.

(51) Int. Cl. A61B 17/02

(52) U.S. Cl. CPC A61B 17/0231; A61B 2017/00862
USPC 600/236

(57) ABSTRACT

An iris retractor assembly (10) including a plurality of iris grabbing hooks (12) disposed or formed at a distal end of one or more slender elements (14) which are pivoted about a pivot member (16), characterized by pincer interface members (18) located at proximal ends (20) of the slender elements (14), the pincer interface members (18) including posts that jut out of the proximal ends (20) perpendicular to a plane of the slender elements (14) and parallel to a pivot axis of the pivot member (16).

### FIELD OF THE INVENTION

The present invention relates generally to iris retractor assemblies used in ophthalmic surgical procedures.

### BACKGROUND OF THE INVENTION

There are various ophthalmic procedures that require the dilation of the pupil. For example, a lens with a cataract is typically removed from the eye by phacoemulsification. This procedure breaks up the lens typically with an ultrasonically driven tool. The tool has an aspiration port that aspirates the broken lens material from the patient's ocular-chamber. It is desirable to extend the pupil during phacoemulsification to provide the surgeon with a wide view of the lens. One technique for extending the pupil includes pulling back or retracting the iris with what is referred to as an iris retractor, and holding the iris at its outer edges.

PCT application WO/2011/053945 (PCT/US2010/055026) of the present assignee describes an iris retractor that has iris grabbing hooks disposed or formed at a distal end of slender elements. A proximal handle is at a proximal end of the slender elements. The slender elements resiliently move between retracted and expanded positions by manipulation of the slender elements. In the retracted position, the hooks are close to one another and the slender elements are close to one another. In the expanded position, the hooks are separate and spaced apart from each other and distal portions of the slender elements are separate and spaced apart from each other. The surgeon inserts the slender elements in the retracted position through a small incision near the limbus of the eye, manipulates the handle to move the slender elements to the expanded position, and grasps and retracts a portion of the iris with the hooks. The incision for insertion of the slender elements can be made at a different position than an incision for phacoemulsification.

### SUMMARY OF THE INVENTION

The present invention seeks to provide further iris retractor assemblies, as is described more in detail hereinbelow.

There is thus provided in accordance with an embodiment of the present invention an iris retractor assembly including a plurality of iris grabbing hooks disposed or formed at a distal end of one or more slender elements which are pivoted about a pivot member, characterized by pincer interface members located at proximal ends of the slender elements, the pincer interface members including posts that jut out of the proximal ends perpendicular to a plane of the slender elements and parallel to a pivot axis of the pivot member.

### BRIEF DESCRIPTION OF THE DRAWINGS

The present invention will be understood and appreciated more fully from the following detailed description taken in conjunction with the drawings in which:

FIGS. 1A and 1B are simplified perspective illustrations of an iris retractor assembly, respectively before and after grasping with pincers, constructed and operative in accordance with an embodiment of the present invention;

FIGS. 2 and 3 are simplified perspective illustrations of iris retractor assemblies, constructed and operative in accordance with other embodiments of the present invention;

FIGS. 4A and 4B are simplified perspective and exploded illustrations, respectively, of an iris retractor assembly having a friction pivot member, constructed and operative in accordance with an embodiment of the present invention;

FIGS. 5A -5C are simplified perspective and exploded illustrations, respectively, of an iris retractor assembly having an incremental locking pivot member, constructed and operative in accordance with an embodiment of the present invention;

FIGS. 6A-6C are simplified perspective illustrations of a holder for any of the iris retractor assemblies of the invention, respectively with the iris retractor in the holder, being released from the holder and lifted from the holder, in accordance with an embodiment of the present invention;

FIG. 7A is a simplified perspective illustration of an iris retractor assembly, constructed and operative in accordance with another embodiment of the present invention;

FIGS. 7B-7C are simplified perspective illustrations of further variants of iris grabbing hooks, in accordance with other embodiments of the present invention;

FIGS. 7D-7E are simplified perspective illustrations of further possible features of the iris retractor, in accordance with other embodiments of the present invention;

FIGS. 8A and 8B are simplified perspective illustrations of using a pincers to grasp the iris retractor of FIGS. 7A-7E;

FIG. 8C is a simplified illustration of a pair of iris retractors in place and used to pull back or retract the iris, in accordance with an embodiment of the present invention;

FIGS. 9A-9F are simplified perspective illustrations of an iris retractor assembly, constructed and operative in accordance with another embodiment of the present invention; and

FIGS. 10A-10D are simplified perspective illustrations of a double biasing member for the iris retractor, constructed and operative in accordance with another embodiment of the present invention.

### DETAILED DESCRIPTION OF EMBODIMENTS

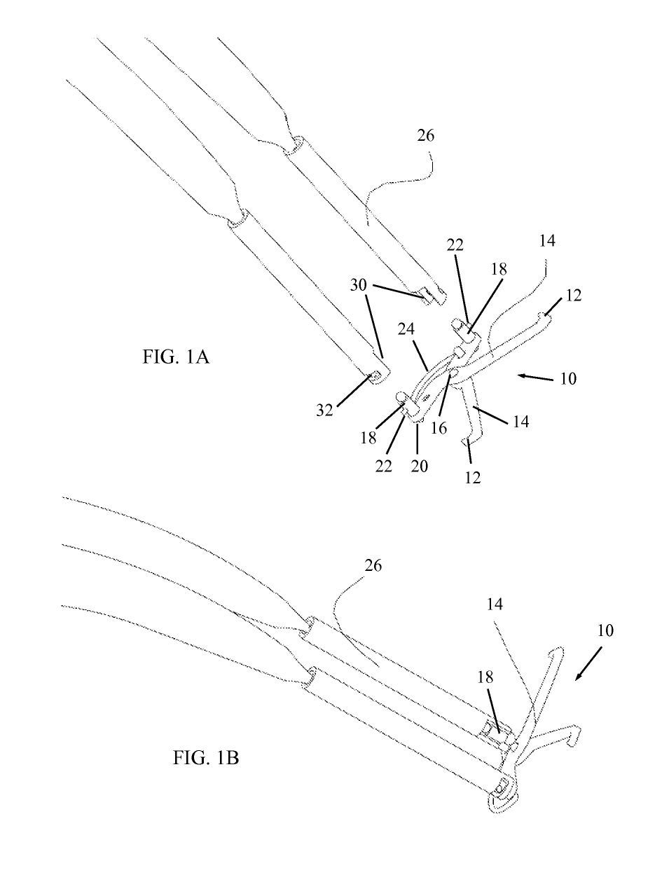

Reference is now made to FIGS. 1A and 1B, which illustrate an iris retractor assembly 10 (also simply referred to as iris retractor 10), constructed and operative in accordance with a non-limiting embodiment of the present invention.

Iris retractor 10 includes a plurality of iris grabbing hooks 12 disposed or formed at a distal end of one or more slender elements 14. In the illustrated embodiment, there are two slender elements 14. The slender elements 14 are pivoted about a pivot member 16. Pincer interface members 18 are located at proximal ends 20 of slender elements 14. Pincer interface members 18 may include, without limitation, posts that jut out of proximal ends 20 perpendicular to the plane of the slender elements 14 and parallel to the pivot axis of pivot member 16. The posts may be provided with lugs 22, perpendicular to the longitudinal axis of the post.

The slender elements 14 may be biased against each other by means of a biasing member 24, such as a springy, resilient wire, band, leaf spring and the like, connected to proximal ends 20 of slender elements 14.

Pincers 26 may be provided to grasp iris retractor 10. Pincers 26 have hollow distal ends 30 formed with a lateral groove 32. When grasping iris retractor 10 with pincers 26, the hollow distal ends 30 go over pincer interface members 18 and lugs 22 are firmly received in grooves 32, as seen in FIG. 1B. Alternatively, the distal ends do not have to be hollow and may be formed with grooves or notches for interfacing with pincer interface members 18.

The iris retractor assembly 10 may be constructed of a medically safe metal or plastic. The iris retractor assembly 10 may be employed similarly to the iris retractors of PCT application WO/2011/053945.

Instead of the posts being on the iris retractor, alternatively the posts may be on the pincers which fit into corresponding recesses or apertures in the iris retractor.

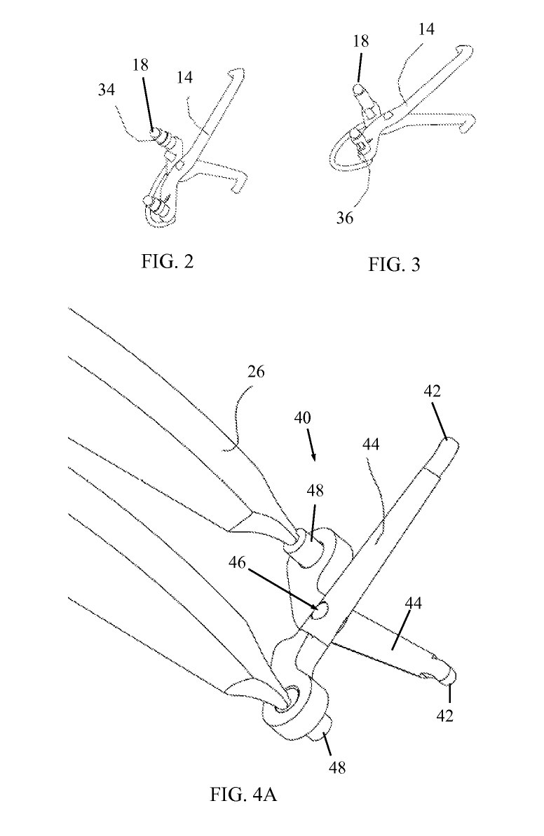

Reference is now made to FIGS. 2 and 3, which illustrate other embodiments of iris retractor assemblies. In FIG. 2, sleeves 34 are fitted over the pincer interface members 18 to enhance the connection between the pincers 26 and the pincer interface members 18. Without limitation, sleeves 34 may simply increase the diameter of members 18 for a better fit; additionally or alternatively, the sleeves 34 may be friction sleeves, which may be made of rubber or other suitable elastomers.

In FIG. 3, the pincer interface members 18 are formed with lateral grooves 36 and the pincers may have corresponding lugs (not shown) for fitting in the grooves 36.

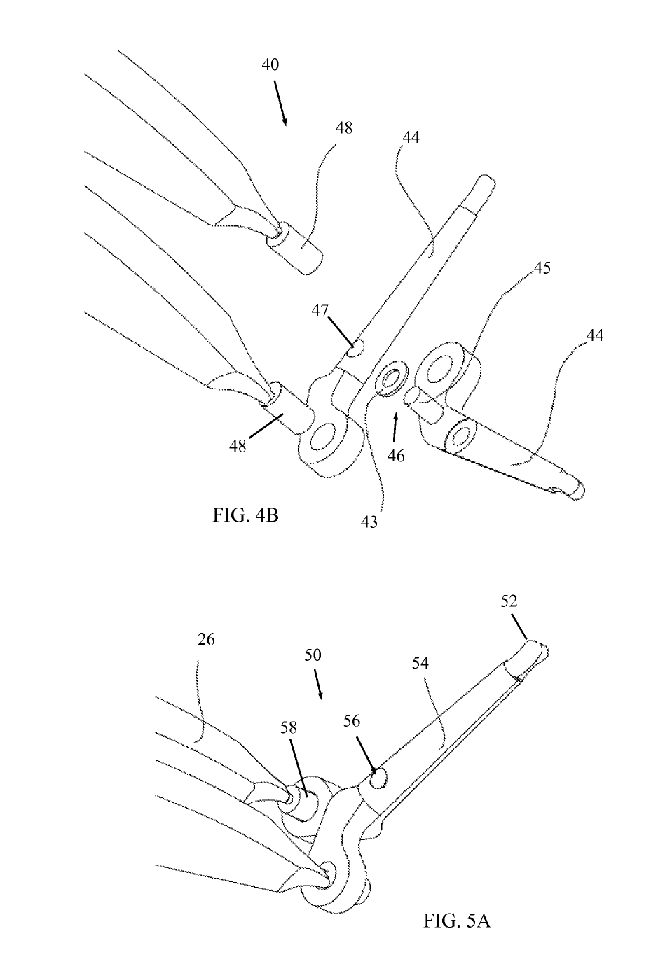

Reference is now made to FIGS. 4A and 4B, which illustrate an iris retractor assembly 40, constructed and operative in accordance with another embodiment of the present invention.

Iris retractor 40 includes a plurality of iris grabbing hooks 42 disposed or formed at a distal end of one or more slender elements 44. The slender elements 44 are pivoted about a friction pivot member 46. Pincer interface members 48 are located at proximal ends of slender elements 44. Pincer interface members 48 may include, without limitation, posts that jut out (in opposite directions, as opposed to the embodiment of FIG. 1A where they jut out in the same direction) of the proximal ends perpendicular to the plane of the slender elements 44 and parallel to the pivot axis of friction pivot member 46. As seen in FIG. 4B, friction pivot member 46 may include an 0-ring or washer 43 mounted on a rod 45 that protrudes from one of slender elements 44 and passes through a hole 47 formed in the other slender element 44.

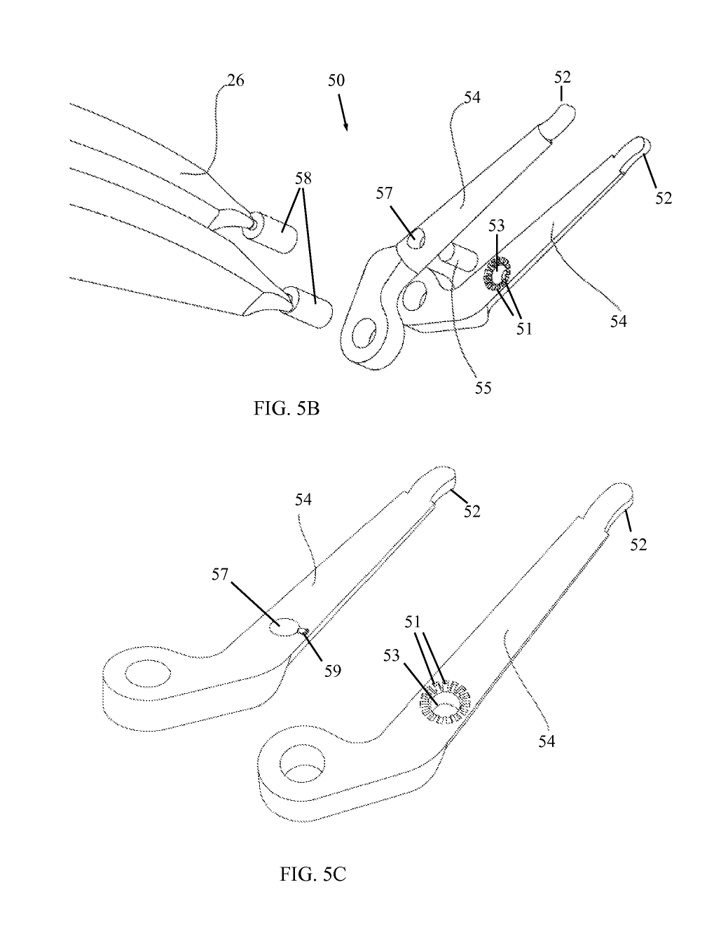

Reference is now made to FIGS. 5A-5C, which illustrate an iris retractor assembly 50, constructed and operative in accordance with another embodiment of the present invention.

Iris retractor 50 includes a plurality of iris grabbing hooks 52 disposed or formed at a distal end of one or more slender elements 54. The slender elements 54 are pivoted about an incremental locking pivot member 56. Pincer interface members 58 of the pincer are placed in proximal ends of slender elements 54. As seen in FIG. 5B, the incremental locking pivot member may include a plurality of grooves 51 circumferentially spaced about a hole 53 (which may or may not be blind) formed in one of slender elements 54. A rod 55 projects from a hole 57 in the other slender element 54 and fits in hole 53. A tab 59 is formed on the slender elements 54 with hole 57. Tab 59 ratchets with grooves 51 to make the incremental locking pivot member.

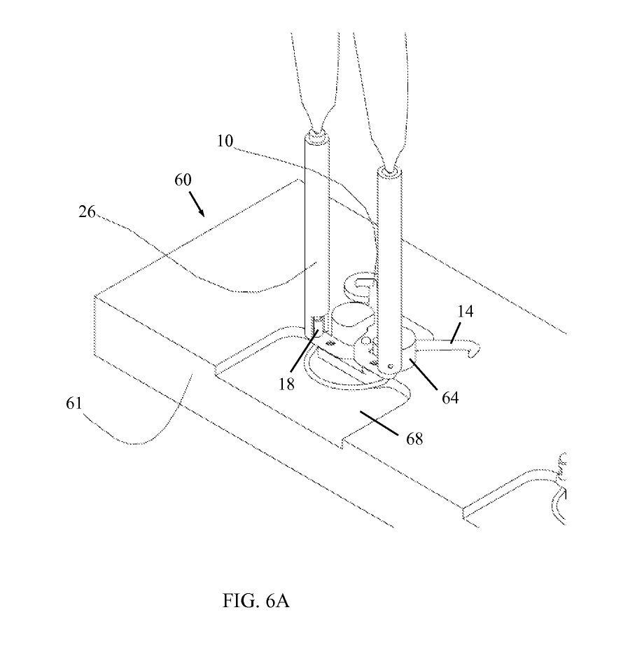

Reference is now made to FIGS. 6A-6C, which illustrate a holder 60 for any of the iris retractor assemblies of the invention, in accordance with an embodiment of the present invention. The holder greatly simplifies handling and manipulation of the iris retractor in preparation for the surgical procedure.

As seen best in FIG. 6C, holder 60 has a base 61 that includes an iris retractor holding assembly 62. The iris retractor holding assembly 62 includes two pillars 64, each formed with a groove 66. When the iris retractor, such as iris retractor 10, is held in iris retractor holding assembly 62, its slender elements 14 rest against opposing grooves 66, as seen in FIG. 6A. The proximal portion of iris retractor 10 is received in a recess 68 formed on base 61. Pincers 26 can be easily inserted into pincer interface members 18, while iris retractor 10 is held in holder 60, as seen in FIG. 6A. In FIG. 6B, the pincers 26 squeeze the slender elements 14 to release iris retractor 10 from holder 60. In FIG. 6C, iris retractor 10 is lifted from holder 60.

As opposed to the embodiment of FIG. 1A, the other embodiments do not have a biasing device and the pincers control the force to open and close the iris retractor, such as with the friction or locking pivot members. These embodiments have a narrower or thinner profile than the embodiment of FIG. 1A.

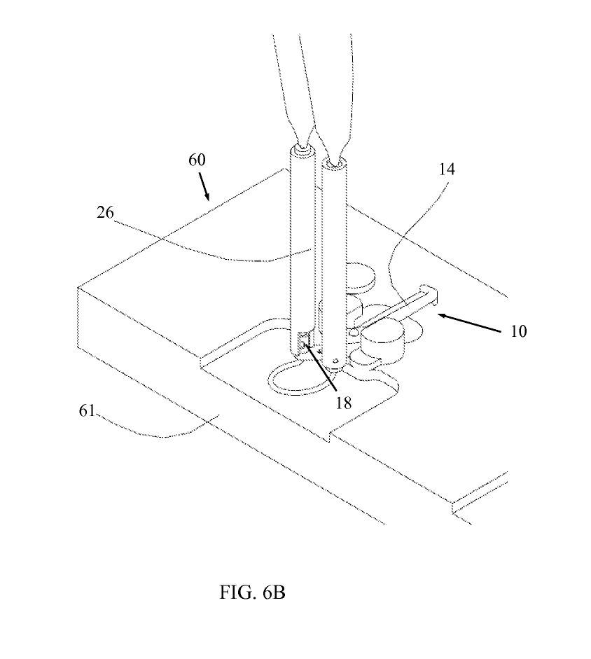

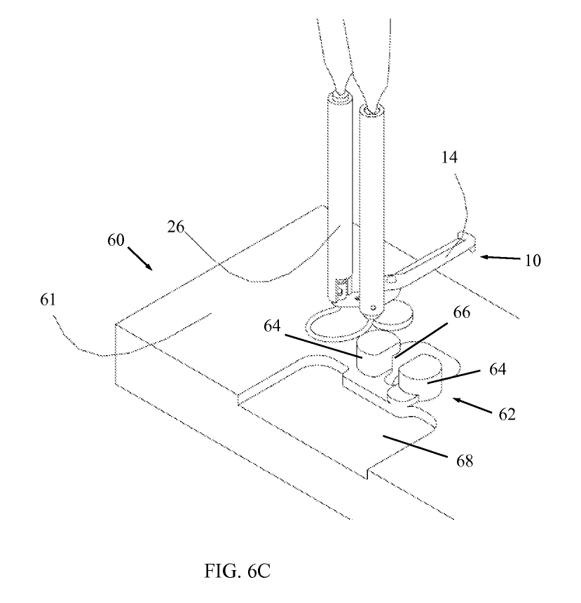

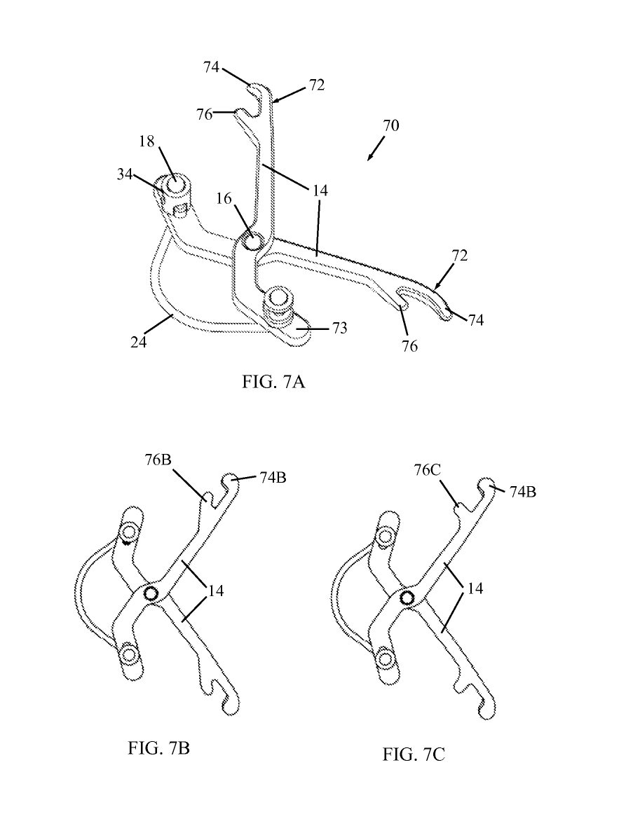

Reference is now made to FIG. 7A, which illustrates an iris retractor assembly 70, constructed and operative in accordance with another embodiment of the present invention.

Iris retractor 70 may be built similarly to iris retractor 10, with like reference numerals designating like elements. Iris retractor 70 differs from iris retractor 10 in its distal iris grabbing hooks 72 disposed or formed at a distal end of one or more slender elements 14. Each grabbing hook 72 includes a distal tooth 74 and a proximal tooth 76. In usage, the iris tissue is held between distal tooth 74 and proximal tooth 76, wherein distal tooth 74 is below the iris and proximal tooth 76 is above the iris.

In the illustrated embodiment of FIG. 7A, distal tooth 74 curves outwardly (outwardly in the sense of outwards from an imaginary centerline that passes through pivot 16 and the middle of biasing member 24) and proximal tooth 76 is tilted towards distal tooth 74. Both distal tooth 74 and proximal tooth 76 preferably have rounded ends. The pincers (or forceps) positioning surface 73 is clearly seen in the drawings from which the pincer interface members 18 protrude.

Reference is now made to FIGS. 7B-7C, which illustrate further variants of the iris grabbing hooks. In the illustrated embodiment of FIG. 7B, a distal tooth 74B is a rounded protuberance, wider than the width of slender element 14. A proximal tooth 76B curves slightly outwardly. In the illustrated embodiment of FIG. 7C, distal tooth 74B is the same as in FIG. 7B, whereas a proximal tooth 76C protrudes generally perpendicularly from slender element 14. These are just some of many other possible configurations of the grabbing hooks.

Reference is now made to FIGS. 7D-7E, which illustrate further possible features of the iris retractor 70. In this embodiment, slender element 14 has one or more bends 77 at any position along the length of slender element 14. Bend 77 can be in any direction, and may be tailored for a particular situation to suit the needs of the patient. Another option is at least one spring stop 78 (FIG. 7E), which is arranged to abut against biasing member 24, thereby limiting the opening angle of iris retractor 70.

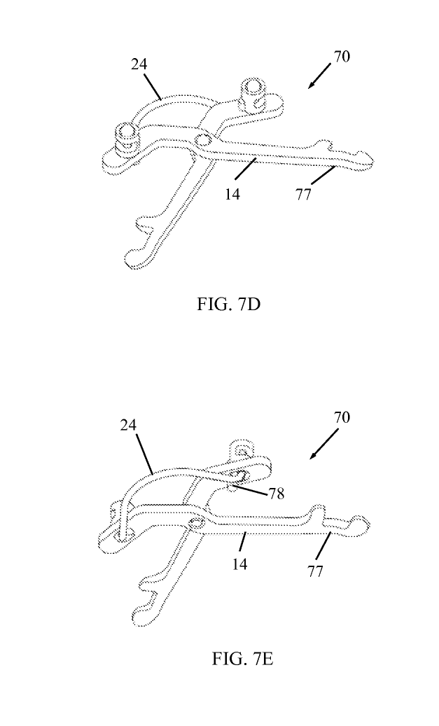

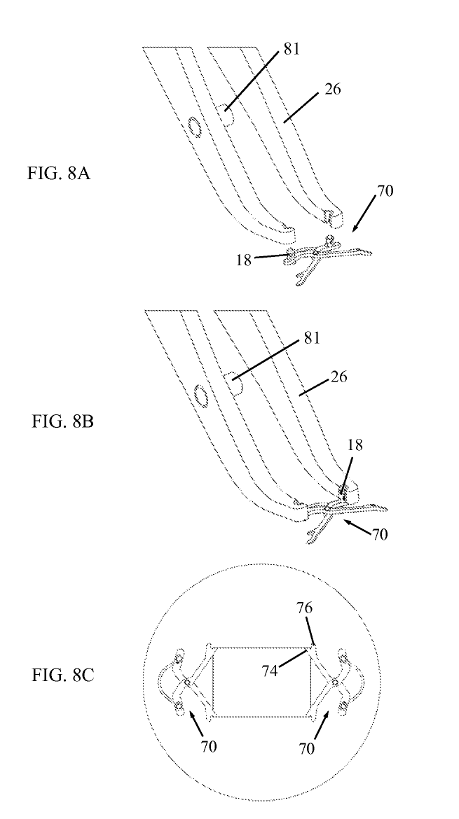

FIGS. 8A and 8B illustrate using a pincers 26 to grasp iris retractor 70, similarly as described above for iris retractor 10. Pincers 26 are provided with a pincers closure limiter 81, such as but not limited to, a pin or other protrusion that protrudes out of one of the pincer arms and is arranged to abut against the other pincers arm to limit the pincers closure. FIG. 8C illustrates a pair of iris retractors 70 in place and used to pull back or retract the iris.

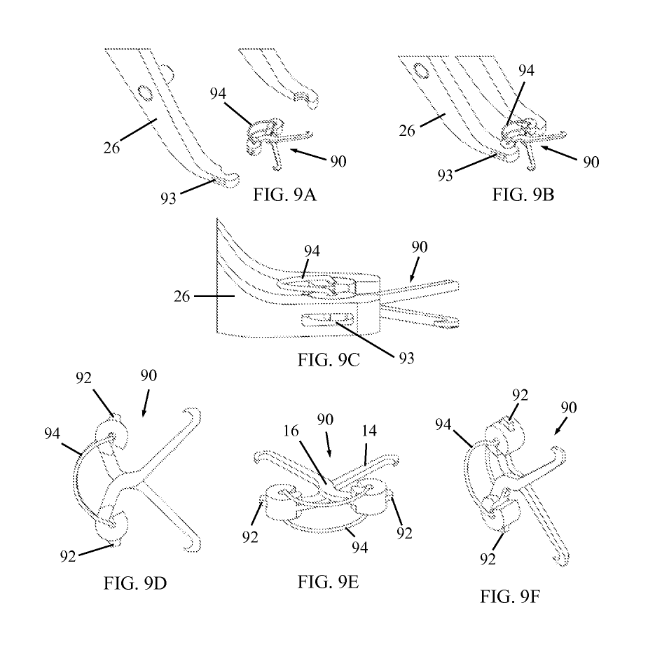

Reference is now made to FIGS. 9A-9F, which illustrate an iris retractor assembly 90, constructed and operative in accordance with another embodiment of the present invention. Iris retractor 90 may be built similarly to iris retractor 10 or 70, with like reference numerals designating like elements. Iris retractor 90 differs from the previously described iris retractors in its use of protruding ears 92 (FIGS. 9D-9F) and a double biasing member 94 (these features may of course be incorporated in any of the iris retractors of the invention). The protruding ears 92 are received in correspondingly formed notches or openings 93 (FIGS. 9A-9C) formed in pincers 26. This may help ensure a good grip on the retractor. The double biasing member 94 provides a balanced and centered (symmetric) spring force on the slender elements 14 of the iris retractor. In other words, the spring force is symmetric about the pivot 16 and this prevents the retractor from twisting or bending due to asymmetric spring forces. This may enable use of plastics to make the retractor, because of the reduced or negligible spring forces on the slender elements 14.

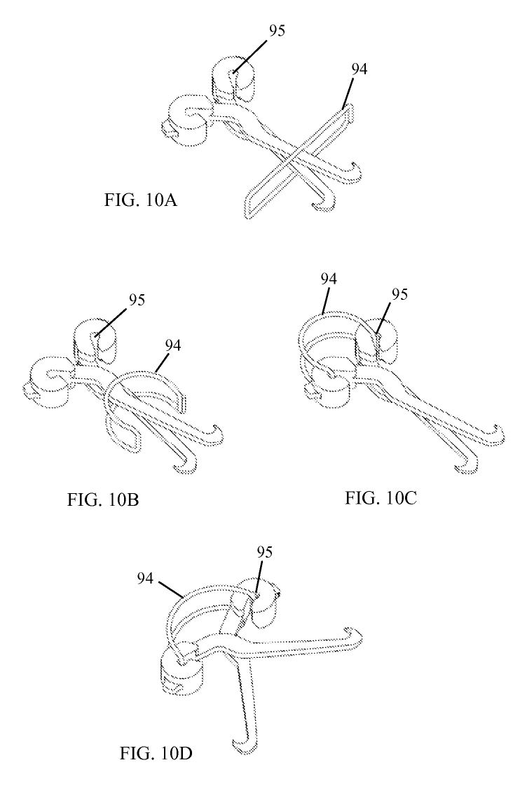

Reference is now made to FIGS. 10A-10D, which show that double biasing member 94 may be constructed as a rectangular or other oblong shape, which is bent and snapped or clicked into place in slots 95 at the proximal ends 20 of slender elements 14.

What is claimed is:

1. An iris retractor assembly (10) comprising:
a plurality of iris grabbing hooks (12) disposed or formed at a distal end of one or more slender elements (14) which are pivoted about a pivot member (16);
characterized by pincer interface members (18) located at proximal ends (20) of said slender elements (14), said pincer interface members (18) comprising posts that jut out of said proximal ends (20) perpendicular to a plane of said slender elements (14) and parallel to a pivot axis of said pivot member (16)

2. The iris retractor assembly (10) according to claim 1, wherein said posts comprise lugs (22) perpendicular to a longitudinal axis of the post.

3. The iris retractor assembly (10) according to claim 1, wherein said slender elements (14) are biased against each other by means of a biasing member (24) at said proximal ends (20) of said slender elements (14).

4. The iris retractor assembly (10) according to claim 1, wherein sleeves (34) are fitted over said pincer interface members (18).

5. The iris retractor assembly (10) according to claim 1, wherein said pincer interface members (18) are formed with lateral grooves (36).

6. The iris retractor assembly (40) according to claim 1, wherein said slender elements (44) are pivoted about a friction pivot member (46).

7. The iris retractor assembly (50) according to claim 1, wherein said slender elements (54) are pivoted about an incremental locking pivot member (56).

8. The iris retractor assembly (70) according to claim 1, wherein each of said iris grabbing hooks (72) comprises a distal tooth (74) and a proximal tooth (76).

9. The iris retractor assembly (70) according to claim 8, wherein said distal tooth (74) curves outwardly.

10. The iris retractor assembly (70) according to claim 8, wherein said proximal tooth (76) is tilted towards said distal tooth (74).

11. The iris retractor assembly (70) according to claim 8, wherein said distal tooth (74B) comprises a rounded protuberance, wider than a width of said one or more slender elements (14).

12. The iris retractor assembly (70) according to claim 8, wherein said proximal tooth (76B) curves outwardly.

13. The iris retractor assembly (70) according to claim 8, wherein said proximal tooth (76C) protrudes generally perpendicularly from said one or more slender elements (14).

14. The iris retractor assembly (70) according to claim 1, wherein said one or more slender elements (14) comprise one or more bends (77).

15. The iris retractor assembly (10) according to claim 3, comprising at least one spring stop (78) arranged to abut against said biasing member (24).

16. The iris retractor assembly (10) according to claim 3, wherein said biasing member (24) comprises a double biasing member (94).
